SEQUENCE LISTING (34 sequences)

SEQ ID NO: 1 - DNA, 31 bp, synthetic construct
  source 1..31: mol_type = other DNA
ggaattccatatggctcgtgcaccgcgttct

SEQ ID NO: 2 - DNA, 27 bp, synthetic construct
  source 1..27: mol_type = other DNA
cccaagcttccgtccggccggtcggct

SEQ ID NO: 3 - DNA, 27 bp, synthetic construct
  source 1..27: mol_type = other DNA
cgcgagctcgctcagaacttcctcgcc

SEQ ID NO: 4 - DNA, 27 bp, synthetic construct
  source 1..27: mol_type = other DNA
gaagttctgagcgagctcgcgccgcgc

SEQ ID NO: 5 - DNA, 27 bp, synthetic construct
  source 1..27: mol_type = other DNA
cgcgagctctgccagaacttcctcgcc

SEQ ID NO: 6 - DNA, 27 bp, synthetic construct
  source 1..27: mol_type = other DNA
gaagttctggcagagctcgcgccgcgc

SEQ ID NO: 7 - DNA, 27 bp, synthetic construct
  source 1..27: mol_type = other DNA
cgcgagctcggtcagaacttcctcgcc

SEQ ID NO: 8 - DNA, 27 bp, synthetic construct
  source 1..27: mol_type = other DNA
gaagttctgaccgagctcgcgccgcgc

SEQ ID NO: 9 - DNA, 27 bp, synthetic construct
  source 1..27: mol_type = other DNA
cgcgagctcttccagaacttcctcgcc

SEQ ID NO: 10 - DNA, 27 bp, synthetic construct
  source 1..27: mol_type = other DNA
gaagttctggaagagctcgcgccgcgc

SEQ ID NO: 11 - DNA, 27 bp, synthetic construct
  source 1..27: mol_type = other DNA
cgcgagctcacccagaacttcctcgcc

SEQ ID NO: 12 - DNA, 27 bp, synthetic construct
  source 1..27: mol_type = other DNA
gaagttctgggtgagctcgcgccgcgc

SEQ ID NO: 13 - DNA, 27 bp, synthetic construct
  source 1..27: mol_type = other DNA
cgcgagctctaccagaacttcctcgcc

SEQ ID NO: 14 - DNA, 27 bp, synthetic construct
  source 1..27: mol_type = other DNA
gaagttctggtagagctcgcgccgcgc

SEQ ID NO: 15 - DNA, 27 bp, synthetic construct
  source 1..27: mol_type = other DNA
gagctctcggaaaacttcctcgcccgc

SEQ ID NO: 16 - DNA, 27 bp, synthetic construct
  source 1..27: mol_type = other DNA
gaggaagttttccgagagctcgcgccg

SEQ ID NO: 17 - DNA, 27 bp, synthetic construct
  source 1..27: mol_type = other DNA
gagctctcgaacaacttcctcgcccgc

SEQ ID NO: 18 - DNA, 27 bp, synthetic construct
  source 1..27: mol_type = other DNA
gaggaagttgttcgagagctcgcgccg

SEQ ID NO: 19 - DNA, 27 bp, synthetic construct
  source 1..27: mol_type = other DNA
gagctctcgagaaacttcctcgcccgc

SEQ ID NO: 20 - DNA, 27 bp, synthetic construct
  source 1..27: mol_type = other DNA
gaggaagtttctcgagagctcgcgccg

SEQ ID NO: 21 - DNA, 27 bp, synthetic construct
  source 1..27: mol_type = other DNA
gagctctcgcacaacttcctcgcccgc

SEQ ID NO: 22 - DNA, 27 bp, synthetic construct
  source 1..27: mol_type = other DNA
gaggaagttgtgcgagagctcgcgccg

SEQ ID NO: 23 - DNA, 27 bp, synthetic construct
  source 1..27: mol_type = other DNA
tcgcagaacgcactcgcccgccgggcc

SEQ ID NO: 24 - DNA, 27 bp, synthetic construct
  source 1..27: mol_type = other DNA
gcgggcgagtgcgttctgcgagagctc

SEQ ID NO: 25 - DNA, 27 bp, synthetic construct
  source 1..27: mol_type = other DNA
tcgcagaaccacctcgcccgccgggcc

SEQ ID NO: 26 - DNA, 27 bp, synthetic construct
  source 1..27: mol_type = other DNA
gcgggcgaggtggttctgcgagagctc

SEQ ID NO: 27 - DNA, 27 bp, synthetic construct
  source 1..27: mol_type = other DNA
tcgcagaacctgctcgcccgccgggcc

SEQ ID NO: 28 - DNA, 27 bp, synthetic construct
  source 1..27: mol_type = other DNA
gcgggcgagcaggttctgcgagagctc

SEQ ID NO: 29 - DNA, 27 bp, synthetic construct
  source 1..27: mol_type = other DNA
tcgcagaactggctcgcccgccgggcc

SEQ ID NO: 30 - DNA, 27 bp, synthetic construct
  source 1..27: mol_type = other DNA
gcgggcgagccagttctgcgagagctc

SEQ ID NO: 31 - DNA, 27 bp, synthetic construct
  source 1..27: mol_type = other DNA
tcgcagaactacctcgcccgccgggcc

SEQ ID NO: 32 - DNA, 27 bp, synthetic construct
  source 1..27: mol_type = other DNA
gcgggcgaggtagttctgcgagagctc

SEQ ID NO: 33 - DNA, 46 bp, synthetic construct
  source 1..46: mol_type = other DNA
ggaattctaatacgactcactatagagagactcggtgaaattatag

SEQ ID NO: 34 - DNA, 28 bp, synthetic construct
  source 1..28: mol_type = other DNA
cgggatcctctcgtactaaggacagctc

(19) United States
(12) Patent Application Publication
(10) Pub. No.: US 2023/0130352 A1
(43) Pub. Date: Apr. 27, 2023

(54) METHOD OF SCREENING INHIBITORS OF ANTIBIOTIC RESISTANCE AND INHIBITORS OBTAINED THEREBY

(71) Applicant: INDUSTRY-UNIVERSITY COOPERATION OF USW, Hwaseong-si Gyeonggi-do (KR)

(72) Inventors: Hyung Jong Jin, Seoul (KR); Hak Jin Lee, Seoul (KR)

(21) Appl. No.: 17/812,747

(22) Filed: Jul. 15, 2022

(30) Foreign Application Priority Data
Jul. 15, 2021 (KR) 10-2021-0093127

(51) Int. Cl. G16C 20/50; C12Q 1/6876; C12Q 1/6811; G16C 20/30; C07H 21/02

(52) U.S. Cl. CPC G16C 20/50; C07H 21/02; C12Q 1/6811; C12Q 1/6876; G16C 20/30; C12Q 2600/154; C12Q 2600/158

(57) ABSTRACT

Disclosed is a method of screening an inhibitor of an erythromycin ribosome methylation (Erm) protein that induces macrolide-licosamide-streptogramin B (MLS_{B}) antibiotic resistance. Specifically, the method of screening an inhibitor that inhibits antibiotic resistance can be used to design and screen inhibitors that prevent or treat resistance to MLS_{B} antibiotics through inhibition or elimination of methylation activity by acting on the shortest motif X, the N-terminal end region (NTER), or a complex structure composed of the same, which are essential for the methylation activity and substrate specificity of Erm proteins that induce resistance to MLS_{B}antibiotics, or a protein structure or a complex structure that forms a substrate attachment site or a ligand attachment site together with the motif X, NTER, or a complex thereof.

### CROSS-REFERENCE TO RELATED APPLICATION

This application claims the benefit of Korean Patent Application No. 10-2021-0093127, filed on July 15,2021, in the Korean Intellectual Property Office, the disclosure of which is incorporated herein in its entirety by reference.

### INCORPORATION BY REFERENCE

This application includes a sequence listing in computer readable form (a “xml” file) that is submitted herewith on an ST.26 XML file named Sequence _Listing .xml, created on Jul. 15, 2022 and 31,057 bytes in size. This sequence listing is incorporated by reference herein.

### BACKGROUND

### 1. Field

The present disclosure relates to a method of screening a substance that inhibits resistance to macrolide-lincosamide-streptogramin B (MLS_{B}) antibiotics and an antibiotic resistance inhibitor obtained thereby, specifically, a method of designing and screening an erythromycin ribosome methylation protein (Erm) inhibitor and an Erm inhibitor obtained thereby.

### 2. Description of the Related Art

The emergence of pathogens resistant to antibiotics reduces the effectiveness of antibiotics, and the so-called superbugs spawned by overuse and misuse of antibiotics have become a great threat to health. A high percentage of clinically isolated pathogens show resistance to macrolide-lincosamide-streptogramin B (MLS_{B}) antibiotics due to erythromycin ribosome methylation (Erm) proteins.

Resistance to antibiotics is achieved by modification of the target site for antibiotics, prevention of antibiotics from accessing to the targets, or chemical modification of antibiotics. For MLS_{B} antibiotics, all of these three resistance mechanisms are found as well, but almost all clinically found resistant strains are shown to have resistance due to target site modification. Modification of the target site is a serious issue, particularly since it leads to the minimum inhibitory concentration which is too high to be overcome by administration of antibiotics.

MLS_{B} antibiotics have different chemical structures, but are classified as macrolide-lincosamide-streptogramin B (MLS_{B}) antibiotics due to the same mechanism by which microorganisms develop resistance thereto. The resistance mechanism against these antibiotics is mediated by the Erm protein. The Erm protein is known to lead to the resistance by inhibiting the MLS_{B} antibiotics from binding by methylation of a specific adenine (A2058) present in 23S rRNA of bacteria. Therefore, inhibitors specific to members of the Erm family are expected to help overcome antibiotic resistance and contribute to achieving therapeutic goals.

Korean Patent Application Publication No. 2001-0090992 discloses a method of enhancing efficacy of antibiotics by inhibiting resistance to MLS_{B} antibiotics antibiotics targeting rRNA of various microorganisms via inhibiton of the activity of Erm proteins, wherein a minimal substrate sequence of rRNA or an amino acid sequence of the N-terminal end region including 55 or more amino acids characteristic of ErmSF is modified. However, there is still a need for target sites for the development of inhibitors capable of effectively inhibiting resistance to MLS_{B} antibiotics.

Accordingly, the present inventors have conducted research to develop inhibitors capable of inhibiting MLS_{B} antibiotic resistance, and found sites in the Erm protein highly conserved and essential for conferring antibiotic resistance.

### SUMMARY

An object of the present disclosure is to provide a method of screening an inhibitor that inhibits resistance to macrolide-lincosamide-streptogramin B (MLS_{B}) antibiotics.

Another object of the present disclosure is to provide a composition for preventing or treating bacterial infection, comprising an inhibitor of resistance to MLS_{B} antibiotics and MLS_{B} antibiotics.

A further object of the present disclosure is to provide a method of preventing or treating a bacterial infection, comprising administering an inhibitor of MLS_{B} antibiotic resistance in combination with an MLS_{B} antibiotics.

### BRIEF DESCRIPTION OF THE DRAWINGS

FIG. 1 shows alignment of an N-terminal end region (NTER) of all erythromycin ribosome methylation (Erm) proteins known to date and a motif X by ESPript 3.0.

FIG. 2 shows results of analysis of in vivo and in vitro activity of a S64 mutant protein. 1: wild-type Erm protein; 2: S64A mutant protein; 3: S64C mutant protein; 4: S64G mutant protein; 5: S64F mutant protein; 6: S64T mutant protein; 7: S64Y mutant protein; 8: control group (no protein).

FIG. 3 shows results of analysis of in vivo and in vitro activity of a Q65 mutant protein. 1: wild-type Erm protein; 2: Q65E mutant protein; 3: Q65N mutant protein; 4: Q65R mutant protein, 5: Q65H mutant protein; 6: control group (no protein).

FIG. 4 shows results of analysis of in vivo and in vitro activity of a F67 mutant protein. 1: wild-type Erm protein; 2: F67A mutant protein; 3: F67H mutant protein; 4: F67L mutant protein; 5: F67W mutant protein; 6: F67Y mutant protein; 6: control group (no protein).

FIG. 5 shows the structure of domain V, a complete substrate known to contain all structural elements for an Erm protein . Domain V is derived from Bacillus subtilis, and consists of 666 nucleotides from C2000 to A2665 in E. coli sequence coordinates (sequence coordinates in parentheses in the figure indicate corresponding positions in Bacillus subtilis). The target adenine of Erm proteins is located at A2058 (based on E. coli accession No. NR_103073; Bacillus subtilis accession No. NR_103037).

FIGS. 6A to 6D show the relative positions of SAH/SAM, target adenine, and S9, Q10, and F12, important residues for catalytic activity, in the ErmC' structure (corresponding to S64, Q65, and F67 in ErmS) . FIG. 6A shows the surface structure of an apo-form of ErmC' with pockets for a cofactor (SAM or SAH) and a target adenine on the left and right, respectively; FIGS. 6B and 6C show a structure in which SAH and adenine are bound; and FIG. 6D shows only 9SQNF12, SAH, and the target adenine. For SAH and the target adenine, N⁶ of the target adenine and the carbon bound thereto are displayed in cyan and magenta, respectively, and remaining C, O, N, and H are colored green, red, blue, and white, respectively For S—Q—F, C is shown in cyan, and the carbons in side chain of N11 are shown in black. It is noteworthy that the surface-exposed groups of the S—Q—F side chains are located close to each other, covering the outside SAM pocket, whereas N is on the opposite side to form the upper inside part of the target adenine binding pocket.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein Accordingly, the embodiments are merely described below, by referring to the figures, to explain aspects of the present description.

An aspect of the present disclosure is a method of screening an inhibitor of an erythromycin ribosome methylation (Erm) protein that induces macrolide-licosamide-streptogramin B (MLS_{B}) antibiotic resistance, comprising: designing a substance that induces a conformational change in a region including the N-terminal end region (NTER) and the shortest motif X adjacent thereto, S—Q—N—F, of an Erm protein; contacting the substance with an Erm protein or a complex of an Erm protein and an RNA substrate and/or a ligand to select a candidate substance that induces a conformational change in a region containing the NTER and the shortest motif X adjacent thereto and/or a site that binds to the substrate or the ligand in the Erm protein or the complex, and identifying whether the conformational change leads to inhibition or elimination of the methylation activity of the Erm protein.

The term “MLS_{B} antibiotic”, used herein, refers to antibiotics of the macrolide, lincosamide, and streptogramin B classes, and is used interchangeably with “MLS_{B}-based antibiotic”. MLS_{B} antibiotics are broad-spectrum antibiotics that act on Gram-negative bacteria such as Klebsiella, and Enterobacter, and Gram-positive bacteria such as Bacillus, and include erythromycin, clarithromycin, tylosine, lincomycin, clindamycin, dalfopristin, etc.

The term “Erm protein”, used herein, refers to a protein that induces resistance to macrolide, lincosamide and streptogramin B antibiotics, and refers to an erythromycin resistance methylase. Erm proteins monomethylate or dimethylate a specific adenine (A2058) present in 23S rRNA of bacteria, thereby inhibiting the binding of MLS_{B} antibiotics and inducing resistance to MLS_{B} antibiotics. Various Erm proteins are known, and the N-terminal end region (NTER) and the shortest motif X adjacent thereto are being studied as potential targets for inhibition of antibiotic resistance.

The term “substance that induces a conformational change in the N-terminal end region (NTER) of an Erm protein and the region including S—Q—N—F, which is the shortest motif X adjacent thereto”, used herein, refers to a substance that induces a conformational change in S—Q—N—F, the shortest motif X and/or an NTER, a region from the N-terminus immediately before a shortest motif X involved in the specific binding of 23S rRNA, a substrate of Erm protein, and ligands acting as cofactors such as S-adenosyl-methionine (SAM) or S-adenosyl-homocysteine (SAH), thereby interfering with or inhibiting binding to a substrate, and/or binding to a ligand, and/or methylation of a target site, or a substance having an activity by itself of interfering with or inhibiting binding to a substrate, and/or binding to a ligand, and/or methylation of a target site in the above-mentioned site or a portion Erm protein and/or Erm and RNA complex and/or Erm and RNA and ligand complex, which cooperatively forms a specific structure with the site.

The shortest motif X of the Erm protein may be generally denoted as G(S/T)-Q-N(H)-F(L/Y), the amino acid in position 1 is S in most Erm proteins, but may be G or T in some Erm proteins, the amino acid in position 3 is N in most Erm proteins, but H in some Erm proteins, and the amino acid in position 4 is F in most Erm proteins, but may be L or Y in some Erm proteins. In addition, in rare cases, the amino acid in position 2 may be W, that in position 3 may be Y, and that in position 4 may be R or P. Herein, the motif X is denoted as SQNF, which is found in most Erm proteins.

The term “design”, used herein, refers to designing, screening, and selecting a structure of a substance capable of binding to or acting on a target site or relevant sites for a desired activity or function, in consideration of the target site and/or the relevant sites; or optimizing a selected candidate substance through the modification by considering interaction with the target site and/or the relevant sites. The design may be performed by using a software that predicts a structure from a compound of a specific formula, an amino acid sequence, or a nucleotide sequence, and predicts or simulates a conformational change due to modification by substitutions, deletions, additions, etc, or contact or binding with other substances For example, in order to design a substance having an activity of inhibiting the methylation activity of an Erm protein, the structure of a target site identified or considered to be related to the methylation activity of the Erm protein or a site related thereto, for example, a site located adjacent to the target site in a three-dimensional structure or involved in binding to a substrate or a ligand may be considered to design or search and select a substance having a structure capable of inhibiting the methylation activity by binding or acting to the target site or the related site thereto.

The term “inhibitor”, used herein, refers to an agent that reduces or eliminates an activity or interaction of an enzyme or a protein, or a substrate interacting therewith, that is, a target substance such as RNA. An inhibitor of an Erm protein refers to a substance that inhibits or prevents antibiotic resistance by inhibiting or eliminating methylation of a target site in an antibiotic by an Erm protein that leads to antibiotic resistance.

The term “cofactor”, used herein, refers to a substance that donates a methyl group for methylation activity of an Erm protein, for example, S-adenosyl-methionine (SAM), and is herein used interchangeably with “ligand”.

A conformational change refers to changes to form a structure required for a protein or an enzyme to selectively and specifically bind and interact with its ligand or substrate, and refers to change from a structure of a protein or an enzyme in the absence of a ligand and/or a substrate to a structure for selective and specific interaction with the ligand and/or substrate by binding thereto, or to a cofactor for such binding. Due to this conformational change, adjacent amino acids in the primary structure may be arranged on opposite sides of the protein Conformational changes include a conformational change induced by the binding or acting of inhibitors of the subject protein, enzyme or substrate to the corresponding site and related sites.

Antibiotic resistance caused by an Erm protein is to prevent an antibiotic from binding to the target site by methylating a specific adenine in 23S rRNA, which is the target site of the antibiotic, and the antibiotic resistance may be inhibited by inhibiting or eliminating the activity of the Erm protein of methylating adenine at the target site. To this end, a substance capable of interfering with or inhibiting the binding of an Erm protein and its methylation substrate, rRNA, or a specific domain thereof, for example, domain V, or a ligand, and/or causing reduction or inhibition of methylation activity, and/or a substance capable of inhibiting or removing methylation by inducing a conformational change in the structure to which rRNA or a ligand is bound and/or RNA itself, may be screened as a potential inhibitor of antibiotic resistance, and an antibiotic resistance inhibitor may be selected by measuring methylation activity of the screened substance.

The N-terminal end region (NTER) from the N-terminus of an Erm protein to a position immediately before the shortest motif X and the shortest motif X following the NTER are found to be important sites for an activity of an Erm protein as a methylating agent and thus as target sites for an inhibitor thereof, through the comparison of amino acid sequences of proteins belonging to an Erm protein family, structural analysis of complexes with ligands and/or substrates, mutant production by site-directed mutagenesis, and measurement of activity and analysis of structural characteristics thereof.

Designing a substance that induces a conformational change in the region including the NTER of the Erm protein and the shortest motif X adjacent thereto, S—Q—N—, means designing a substance that affects the binding of the Erm proteins to substrates and/or ligands and/or affects the methylation activity, by inducing a conformational change in the region including the NTER of the Erm protein and the shortest motif X adjacent thereto, S—Q—N—F The conformational change by the substance may lead to inhibition of interaction of the Erm protein with the substrate RNA or inhibition of the methylation reaction, thereby causing resistance.

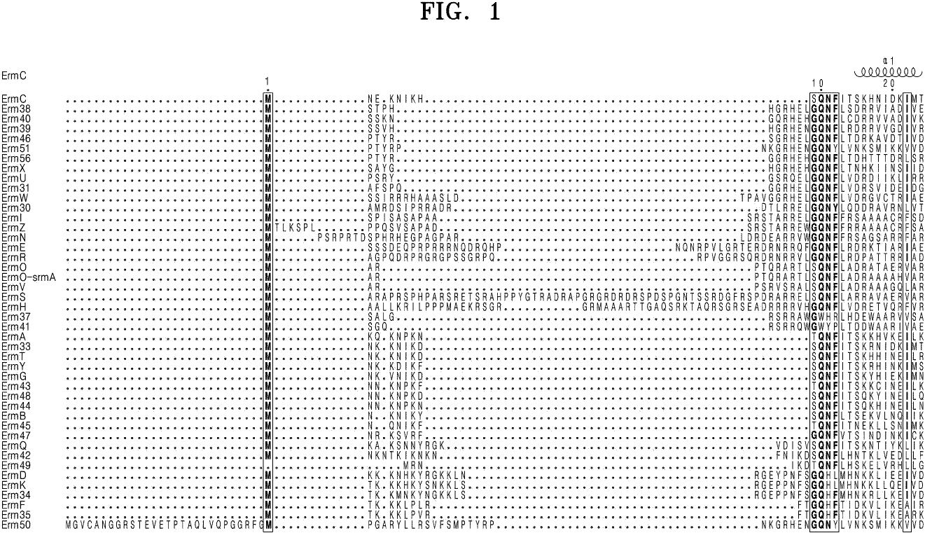

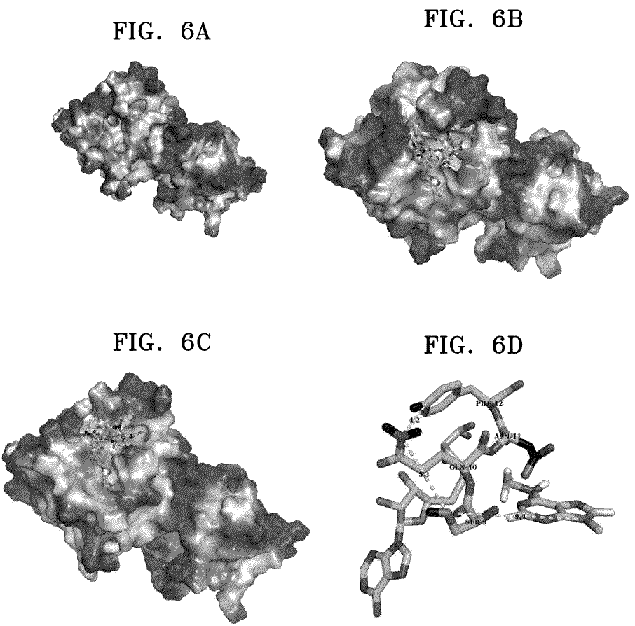

Specifically, as shown in FIG. 1, the NTERs of an Erm protein have various lengths, and the shortest motif X (SQNF) is highly conserved. It was found through structural analysis studies using PyMOL that surface-exposed functional groups of side chains of S, Q, and F, which are important for catalytic activity of Erm proteins, are positioned adjacent to each other, covering the outside of the SAM pocket, and N forms an upper inside part of the target adenine binding pocket (see FIGS. 6A-6D). In the structure where ErmC' is bound to SAH and the target adenine, the side chains of S, Q, and F do not interact with SAH and, in particularl, the hydroxymethyl group of S, the amine of the carbamoyl group of Q, and position 3, 4, and 5 in the benzene ring of the phenylmethyl group of F face the solvent and form an outer surface of the SAM (SAH) binding pocket, whereas main chains of S, Q, and F form a part of the inside wall of the SAM binding pocket. The distance between the nitrogen of the carbamoyl group in Q and the oxygen of the hydroxyl group in S was 5.3 Å and the distance between the nitrogen of the carbamoyl group and C4 carbon of the phenyl group of F was 4.2 Å. This structure may contribute to the binding of the Erm protein to a substrate RNA. On the other hand, the side chain of N forms a part of the inner wall of the target adenine binding pocket, which suggests contribution to the stabilization of the target adenine. In fact, S—Q—F and N are sequentially adjacent in the primary structure, but their side chains are located on the opposite sides in the tertiary structure. In addition, the side chains of S—Q—F are close to the activation site. Even in the apo form of the enzyme not bound to a ligand, the side chains of Q and F are further separated than in the complex structure, are directed away from SAM to face the solvent, and N is in a disordered state. These residues are located immediately next to the bound SAM within a distance of 6 Å, forming a part of the binding pocket for SAM, and are the only region where the conformation changes with binding of the ligands, presumably to provide fitness for binding to a substrate. That is, SQNF is identified as a key site for ligand binding and substrate selectivity and binding of an Erm protein Interactions with the substrate and conformational change at the site and NTER, connected thereto and is known to play a partly important role in substrate specificity, affect the activity of Erm, and thus, may affect the resulting antibiotic resistance.

An Erm protein methylates an adenine in 23S rRNA and interferes with attachment of antibiotics, thereby interfering with its antibiotic action.

In an embodiment of the present disclosure, the selecting of a candidate substance may be performed in silico.

In an embodiment of the present disclosure, the selecting of a candidate substance may be performed by using one or more of X-ray crystallography, cryogenic electron microscopy (cryo-EM), and nuclear magnetic resonance (NMR) spectroscopy.

In order to select a substance that acts on an Erm protein or a complex of an Erm protein and an RNA substrate and/or a cofactor to interfere with the interaction thereof or cause a conformational change, thereby leading to inhibition or elimination of the methylation activity, a program that generates a three-dimensional structure of a complex of an Erm protein and an RNA substrate or a ligand or a complex of an Erm protein, a substrate and a ligand, for example, a program such as PyMol may be used to generate the structure, or the aforementioned X-ray crystallography, cryo-EM, and NMR spectroscopy may be used to generate the structure of the Erm protein, or the complex thereof; and conformational changes that may be brought about when the test substance is applied thereto may be identified in silico or by using X-ray crystallography, cryo-microscopy, or nuclear magnetic resonance spectroscopy, or a combination thereof. In addition, it may be confirmed whether a conformational change is generated in the NTER of an Erm protein and the shortest motif X adjacent thereto by using a program that simulates binding of a protein and a substrate and resulting conformational change or action.

A candidate substance may be designed, based on the structure of the complex of Erm protein and RNA substrate and/or ligand, to lead to a conformational change by acting thereon, for example, to have a structure that inhibits binding of the NTER or the motif X or NTER/motif X in an Erm protein, or other parts of the protein that act cooperatively to form a specific structure with an RNA substrate and/or a ligand, or induces a change in the structure or position for the binding. The test substance may be a substance that causes a genetic modification leading to amino acid substitutions that change the size or electron density of the side chains of S, N and F of the motif X or the amino acids constituting NTER of the Erm protein, for example, genetic scissors for such modifications.

The candidate substance may be a substance that mimics the NTER and the shortest motif X.

A conformational change due to a structure of a protein and a substrate, or a protein-substrate complex or a protein-substrate-ligand complex structure, and a substance acting thereon may be predicted by: determining the structure of the protein from the amino acid sequence or from analysis by X-ray crystallography, NMR, cryo-EM, etc., and directly analyzing the structure obtained upon application of a substrate and/or a test substance on the protein by using the above analysis method or simulating it using a program.

In an embodiment of the present disclosure, the conformational change may be change in a length of an NTER of the Erm protein or a distance between the Erm protein and the RNA substrate or the ligand

In an embodiment of the present disclosure, the ligand may be SAM.

In an embodiment of the present disclosure, the reduction or elimination of the methylation activity may be determined by an in vitro methylation activity assay, an in vivo antibiotic resistance assay, or a minimal inhibitory concentration (MIC) measurement.

The methylation activity assay may be performed by reacting a candidate substance with a fluorescence-labeled or radio-labeled ligand, and 23S rRNA, which is a substrate of an Erm protein, or domain V or a fragment thereof; and detecting methylated RNA.

In an embodiment of the present disclosure, the candidate substance may be a compound, a nucleic acid, a peptide or a protein.

In an embodiment of the present disclosure, the candidate substance may be a compound designed to have a structure capable of binding to the NTER of an Erm protein and/or the shortest motif X adjacent thereto.

In an embodiment of the present disclosure, the candidate substance may be a nucleic acid, a peptide, or a protein that forms a structure capable of binding to the NTER of the Erm protein and/or the shortest motif X adjacent thereto, or a nucleic acid encoding the peptide or protein.

In an embodiment of the present disclosure, the inhibitor may be nucleic acids, peptides, or proteins to be delivered via genetic scissors.

In an embodiment of the present disclosure, the candidate substance may cause a conformational change in the shortest motif X of the Erm protein, SQNF by changing the distance between the nitrogen of the carbamoyl group in Q and an oxygen of a hydroxyl group in S, the distance between the nitrogen of the carbamoyl group and C4 carbon of the phenyl group in F, and the distance between the oxygen of the side chain hydroxyl group in S and the 6th nitrogen of the adenine, a methylation target.

In an embodiment of the present disclosure, the method may further include selecting the candidate substance as an inhibitor when the candidate substance is confirmed to inhibit or remove methylation activity.

In an embodiment of the present disclosure, the method may further include applying the candidate substance identified to lead to inhibition or elimination of the methylation activity to an in vivo antibiotic resistance inhibition assay.

The in vivo antibiotic resistance inhibition assay, used herein, may be a method of identifying whether there is a region that becomes transparent or thinned by inhibition of bacterial growth around the site where a filter paper is placed, after placing the filter paper on a plate seeded with bacteria expressing antibiotics resistance by an Erm protein, wherein the filter paper acts as a depot of an antibiotic or antibiotics and resistance inhibitor(s). In addition, the in vivo antibiotic resistance inhibition assay may be a method of measuring minimal inhibitory concentration (MIC).

In an embodiment of the present disclosure, the method may select a candidate substance whose in vitro inhibition or elimination of the methylation activity and in vivo inhibitory activity of antibiotic resistance are confirmed as an Erm protein inhibitor

In an embodiment of the present disclosure, the Erm protein may be ErmC' or ErmS, and the RNA substrate may be 23S rRNA, domain V, or a fragment thereof

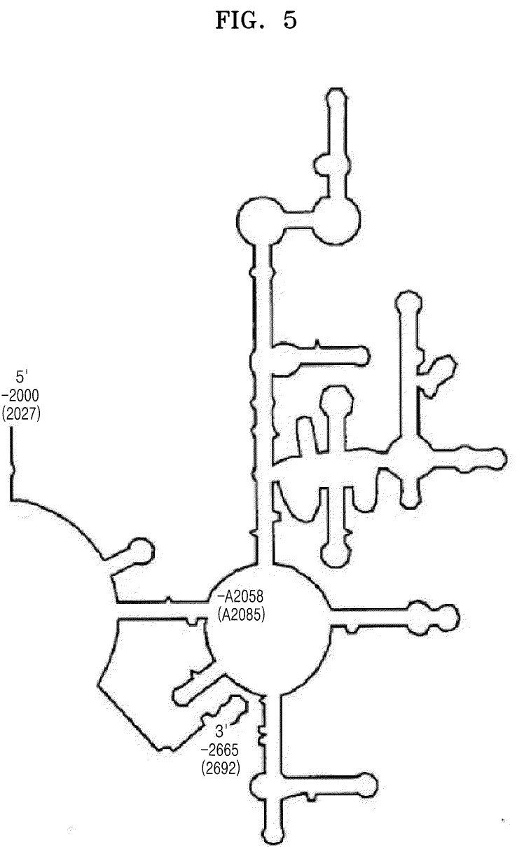

Domain V is a domain known to have all the structural elements necessary to act as a substrate for an Erm protein, and may be derived from microorganisms in which Erm proteins are found, including Bacillus sublilis. Domain V is derived from Bacillus subtilis, and may be a domain consisting of 666 nucleotides from C at position 2000 to A at position 2665 that encode a portion of the 23S rRNA, and A at position 2085 is the target adenine of the Erm protein. The structure is shown in FIG. 5.

Another aspect of the present disclosure provides an inhibitor of an Erm protein, which reduces or eliminates methylation activity of an Erm protein by causing conformational or functional change in the NTER and the shortest motif X adjacent thereto, S—Q—N—F, in the Erm protein.

In an embodiment of the present disclosure, the inhibitor may be one that induces a conformational or activity change of the NTER and/or the shortest motif, by binding to a separate portion which forms a specific structure with the NTER and/or the shortest motif of the Erm protein.

In an embodiment of the present disclosure, the inhibitor may be one that induces amino acid substitution in S—Q—N—F and/or NTER.

In an embodiment of the present disclosure, the inhibitor may be genetic scissors introducing an amino acid substitution in the shortest motif X and/or NTER of an Erm protein.

The term “genetic scissors”, used herein, is used interchangeably with gene editor, and CRISPR, and refers to a nucleic acid-protein complex capable of inducing a desired genetic modification at a specific target location in a genome. Specifically, genetic scissors include a guide RNA capable of recognizing and binding to a target site, and a Cas (CRISPR-associated) protein capable of introducing a desired mutation by cleavage and homologous recombination or heterologous recombination at the target site, and a template nucleic acid fragment for introducing a desired mutation may be further included.

When genetic scissors, which introduce a mutation that inhibits or eliminates methylation activity in the motif X and/or NTER of an Erm protein, are introduced into a bacterium whose antibiotic resistance needs to be addressed, a desired mutation may be introduced into the nucleic acid encoding the Erm protein in the bacterium to reduce or inactivate the Erm protein, and thus, antibiotic resistance may be inhibited.

A guide RNA for introducing a desired mutation into a target site may be designed by using a program well known in the art to which the present disclosure pertains.

In an embodiment of the present disclosure, the substitution may be substitution of S with G, A, C, T, F, Y, or other amino acids, substitution of Q with N, E, R, H, or other amino acids, or substitution of F with A, H, L, Y, W, or other amino acids in the shortest motif X.

In an embodiment of the present disclosure, the substitution may be substitution of S with G, A, C, T, F, or Y, substitution of Q with N, E, R, or H, or substitution of F with A, H, L, Y, or W in the shortest motif X.

The size and charge of the side chain were found important to the activity of the Erm protein by testing the activity following introduction of a mutation in each amino acid in the motif X of the Erm protein, and when a side chain larger than the original side chain was introduced, the activity was found inhibited to suppress resistance, and when a size of the side chain was maintained similarly or decreased, resistance was observed. Specifically, when S in motif X was changed to G, that is, when the size of the side chain was reduced, about 80% of activity was retained, but when the side chain is bigger than that of G, an interaction with a substrate was found to require an exact size of the side chain, electronegativity, acidity, and size of atoms (S64C and S64A). However, when the amino acid is substituted with an amino acid having a side chain larger than that of S at this site, the activity was not observed (S64T, S64F, and S64Y). For Q, only Q is allowed at the position, and when an amino acid other than Q is located at the position, all activity is lost. F67 requires a hydrophobic side chain of an appropriate size (F67A, F67H, F67L, F67Y and F67W). These observations strongly suggest that the amino acids constituting the motif X interact with the substrate, along with the observation that the constituents of motif X exist close to each other in the protein structure, their side chains interact with the solvent, and flexibility thereof is great. In addition, respective interactions of the constituents of motif X have a profound effect on the methylation activity of proteins. In addition, the motif X exists close to the adenine to be methylated, so that it may play a role in orientation of the adenine to be methylated and a cofactor for the methylation. Therefore, when a conformational change that prevents or inhibits an effective interaction between the Erm protein and the substrate occurs by an amino acid substitution or the like, methylation of the target site by the Erm protein cannot occur, thereby suppressing antibiotic resistance.

In an embodiment of the present disclosure, the substitution may be two or more amino acid substitutions in the SQNF motif.

Another aspect of the present disclosure is to provide a composition for inhibiting antibiotic resistance, comprising an MLS_{B} antibiotic resistance inhibitor(s) and an MLS_{B} antibiotic.

Bacteria are generally killed when antibiotics are administered, but emergence, proliferation and spread of mutant bacteria resistant to antibiotics have led to superbugs to which antibiotics are not effective anymore, posing a great threat. An antibiotic resistance inhibitor may be administered in combination with the antibiotic, in order to enhance the efficacy of the antibiotic and to suppress resistance to the antibiotic.

In an embodiment of the present disclosure, the inhibitor of resistance to MLS_{B} antibiotics may be a compound, a nucleic acid, a peptide, or protein that inhibits the methylation activity of an Erm protein

In an embodiment of the present disclosure, the inhibitor of resistance to MLS_{B} antibiotics and the MLS_{B} antibiotics may be administered simultaneously or sequentially.

In an embodiment of the present disclosure, the inhibitor of resistance to MLS_{B} antibiotics and the MLS_{B} antibiotics may be administered at the same administration interval or may be administered at different administration intervals, and the administration duration may be different.

A method of screening an inhibitor that inhibits antibiotic resistance, according to an embodiment of the present disclosure, can be used to design and screen inhibitors that prevent or treat resistance to MLS_{B} antibiotics through inhibition or elimination of methylation activity by acting on the motif X, NTER, or a complex structure composed thereof, which are essential for the methylation activity and substrate specificity of Erm proteins that induce resistance to MLS_{B} antibiotics, or a protein structure or a complex structure that forms a substrate attachment site or a ligand attachment site together with the motif X, NTER, or a complex structure composed thereof

### EXAMPLES

Hereinafter, the disclosure will be described in more detail through examples. However, these examples are intended to illustrate the present disclosure, and the scope of the present disclosure is not limited to these examples.

### Example 1. Screening of Target Site for Inhibition of Methylation Activity of Erm Protein

In this Example, the sequences of Erm proteins were compared to identify a target site for inhibiting antibiotic resistance by an Erm protein. Using ESPript 3.0, the NTER and the shortest motif X of 44 Erm proteins known to date were aligned. The results are shown in FIG. 1. Regions containing the NTER, motif X, and a first alpha helix were aligned. NTER refers to a sequence from the N-terminus to a position immediately before the shortest motif X, which is represented as SQNF. No conserved sequence was detected in NTER, but a high degree of conservation was confirmed in motif X.

The length of NTER from the amino acid in position 1 to a position immediately before the motif X varied from 63 amino acids (Erm49), and no consensus was detected among NTERs. However, high conservation of the amino acid sequence was identified in the region indicated as motif X immediately following it. In this motif, the first amino acid could be represented as G in most Erm proteins, 26 Erms including Erm37 and Erm41, while the amino acid was S in 14 Erm proteins and T in 4 Erm proteins. In position 2 of this motif, Q was found completely conserved, except for Erm37 and Erm41. Erm37 and Erm41 did not have a C-terminal domain and Q was switched to W. Erm37 showed promiscuous methylation activity at adjacent positions in the vicinity of A2058, whereas Erm38 is an A2058-specific dimethylase having a low activity with most rRNAs either monomethylated or unmethylated. The third amino acid in the motif was N in 37 Erms, H in 6 Erms and Y for Erm41. The last as the fourth amino acid in the motif was conserved as F in 38 Erms, L in two Erms, Y in another two Erms, and R and P in Erm37 and Erm41, respectively. Therefore, the motif X was well conserved in Erm proteins

NTER lengths varied among Erm proteins. When loops 1 and 12 of BsKsgA, which has sequence homology with Erm, were swapped with ErmC’, substrate specificity was identified to be switched partially from the substrate specificity of KsgA to the substrate specificity of ErmC'. In Erm proteins, loop 1 could be an NTER or a part of it, or it could be located immediately adjacent to the motif X. Therefore, loop 1 may contribute to the selective and specific recognition of the substrate for Erm. For function of Erm proteins, the surface-exposed side chains of S—Q—F in the motif X exist very close to each other and relatively strict and/or absolute requirements for their identity should be met. This region encompassing the NTER and the motif X is intrinsically flexible, and is in close proximity to the activation site where methylation occurs (see FIGS. 6A to 6D). In addition, upon binding to SAM and substrate, unique conformational changes in this region are expected. Therefore, this site is thought to interact specifically with the substrate and has a significant effect on the activity, and thus, the site may be a potential target site for the development of inhibitors.

### Example 2. Inhibition of Methylation Activity by Mutation of Erm Protein

### 2-1. Expression of Mutant Protein in E. Coli

### Materials

E. coli DH5a (Promega, Madison, Wl, USA) and BL21 (DE3) (Novagen, Madison, WI, USA) were used for general cloning and expression of Hiₛ₆-tagged Erm proteins, respectively. The sequence of domain V of 23S rRNA used in this Example was cloned from Bacillus sublilis BD170 and exhibited three differences compared with the one presented on the Gutell Lab’s Comparative RNA Website (CRW site: http.//www.ma.icmb.utexas.edu, accessed on December 2020): two ): mutations (C2203G and U2629A) and one nucleotide deletion (Δ C2473). While 19 identical sequences were identified when searching on GenBank with the sequence used herein as a query, only 2 sequences showed exact sequence identity with the one from the Gutell Lab’s CRW site Restriction enzymes were purchased from New England BioLabs and used according to the manual. LB media and Bacto agar for bacterial culture were purchased from Difco Laboratories, and Taq polymerases and nucleotides were purchased from TaKaRa Shuzo Co. For in vitro transcription, spermine, Triton X-100, and polyethylene glycol (PEG; molecular weight, 8000) were purchased from Sigma Chemical Co. T7 RNA polymerase was prepared in-house. His Bind resin was purchased from Novagen, reagents for polyacrylamide gel electrophoresis, such as N,N,N′,N′-tetramethylethylenediamine (TEMED), were purchased from Bio-Rad, and conventional chemicals, such as salts, buffer components, agarose, and antibiotics, were purchased from Sigma Chemical Co.

### Site-Directed Mutagenesis and Construction of Expression Vector

The expression vector (pHJJ105) and E. coli strain (E. coli HJJ105) overexpressing the wild-type ErmS were obtained from previous studies of the present inventor (ACS Omega 2017, 2, 8129-8140, Protein Expr Purif. 2002, 25, 149-159). Site-directed mutagenesis was carried out by a SOE-PCR(splicing by overlap extension PCR) To obtain a DNA fragment for S64A covering the 5′-end region, PCR was performed using the pHJJ105 plasmid DNA as a template and oligo-1 and oligo-4 as forward and reverse primers, respectively. To obtain a DNA fragment for S64A covering the 3′-end region, PCR was performed using the pHJJ105 plasmid DNA as a template and oligo-3 and oligo-2 as forward and reverse primers, respectively. Thereafter, two DNA fragments obtained for the 5′- and 3′-end regions, respectively and having the overlapping region harboring the mutated site (S64A) in common were combined, and PCR was carried out using oligo-1 and oligo-2 as forward and reverse primers, respectively, to obtain a whole ermS gene having the S64A mutation The sequences of the primers were modified to include a restriction site (oligo- 1) for Ndel (5-catatg-3′), overlapping the initiation Met codon, and a restriction site for HindIII(5′-aagcct-3). The resultant PCR product was treated with Ndel and HindIII restriction enzymes, and the DNA fragments containing the S64A mutation were ligated to the pET23b Ndel-HindIII sites. The cloned gene was sequenced to confirm the target sequence and frame. Expression vectors for all the other mutant genes were also constructed in the same way. All the primers for cloning of mutant ermS-encoding DNA fragments are summarized in Tables 4 to 7.

### Protein Expression and Purification

E. coli BL21(DE3) cells transforemd with wild type ermS or mutant ermS genes in the pET-23b vector were grown overnight at 37° C. in LB medium supplemented with 100 mg/mL of ampicillin. The culture was transferred to fresh LB medium (10%, v/v) and incubated at 37° C. for another 1.5 hours to reach an A₆₀₀ of 0.8 to 1.0. In order to induce expression, isopropyl-β-D-thiogalactopyranoside (IPTG) was added to a final concentration of 1 mM and incubated at 22° C. for 18 hours. Purification was performed as previously described (Antimicrob. Agents Chemother. 2020, 64, 45). Briefly, cells were collected from 100 mL of culture by centrifugation and resuspended in buffer A (20 mM Tris-HCl [pH 7.0], 500 mM KCI, and 5 mM imidazole). The cells were disrupted by sonication on ice by using a GEX-130 ultrasonic processor (130 W, 20 kHz) (50% amplitude 5-second pulse and 10-second pause for cooling). The total sonication time was 5 minutes. The lysate was centrifuged to remove the cell debris and other insoluble materials, and the supernatant was loaded onto a column containing His Bind resin pre-equilibrated with buffer A. Next, the column was washed extensively with buffer B (20 mM Tris-HCl [pH 7.0], 500 mM KC1, and 100 mM imidazole) to remove unbound or falsely bound proteins, and then, proteins were eluted with buffer C (20 mM Tris-HCl [pH 7.0], 500 mM KC1, and 300 mM imidazole). To remove the imidazole and salts, the eluted protein solution was purified using a PD-10 desalting column, according to GE Healthcare’s manual, and was stored at -20° C. in 20 mM Tris-HCl [pH 7.0], 200 mM KC1, 1 mM EDTA, and 50% glycerol. The protein concentration was determined by the bicinchoninic acid (BCA) protein assay method (Pierce, Rockford, IL, USA).

### Cloning of Bacillus Subtilis Domain V DNA and in Vitro Transcription

To obtain a DNA fragment encoding domain V, PCR was performed using Bacillus subtilis BD170 chromosomal DNA as a template and GGAATTCtaatacgactcactataGAGAGACT CGGTGAAATTATAG (SEQ ID NO: 33) and CGGGATCCTCTCGTACTAAGGACAGCTC (SEQ ID NO: 34) as forward and reverse primers, respectively. The sequences of these primers correspond to nucleotides 2000 to 2021 and 2643 to 2665 in E. coli coordinate (2027 to 2048 and 2670 to 2692 in Bacillus subtilis coordinate). The underlined sequences represent EcoRI and BamHI restriction sites, respectively, and the lowercase nucleotides indicate the T7 promoter sequence. The resultant PCR product was treated with EcoRI and BamHI restriction enzymes, and the DNA fragment containing the domain V was ligated to EcoRI and BamHI treated pUC19. The cloned gene was sequenced to confirm the sequence and frame of the insert. Domain V was transcribed in vitro using a phage T7 RNA polymerase. The plasmid was used as a templates for in vitro transcription, and the plasmid was linearized with BamHI for runoff transcription. After BamHI treatment, five nucleotides were presented as an overhang, three of which were exactly the same as the sequence of domain V. Therefore, in the final transcript product, two nucleotides (UC) derived from a BamHI recognition sequence were additionally provided (668 nt; see FIG. 5). The linearized plasmid was directly used as the template for the synthesis of substrate RNA transcripts. Transcription from the linearized plasmid templates was performed in a 500-µL reaction mixture containing 40 mM Tris-HCl (pH 8.1), 5 mM dithiothreitol (DTT), 1 mM spermine, 0.01% Triton X-100, 80 mg/mL PEG, 25 µg of DNA template, 4 mM ribonucleoside triphosphates (rNTPs), 28 mM MgC1_{2,}and 10 µg of T7 RNA polymerase at 37° C., for 4 hours. After transcription, the transcripts were extracted with phenol-chloroform, precipitated with ethanol, and resuspended in diethyl pyrocarbonate (DEPC)-treated water. The transcripts produced were visualized on 5% 7 M urea-polyacrylamide gel with UV to check the integrity and verify the size. The bands with the correct size were extracted in a Tris-borate(TBE)-EDTA buffer by electrophoresis, precipitated with ethanol, and was dissolved in a self-folding buffer (50 mM HEPES-KOH [pH 7.5], 20 mM magnesium acetate, and 400 mM NH₄Cl), heated at 65° C. for 10 minutes, and then cooled to 37° C. over a period of 90 minutes to complete the self-folding of the RNA transcripts.

### 2-2. In Vivo and in Vitro Analysis of Mutant Protein Activity

The methylation activity and antibiotic resistance inhibitory activity of the mutant Erm proteins obtained in 2-1 were analyzed.

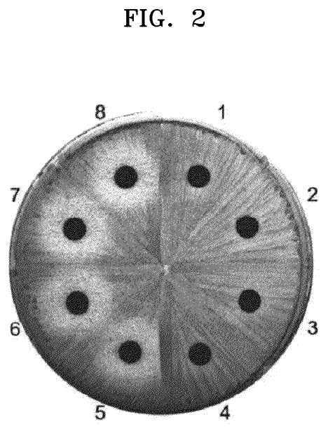

Specifically, in the highly conserved motif X of an Erm protein, S64, Q65 and F67 were mutated to related amino acids, i.e., G, A, C, T, F, and Y (for S64), E, N, R, and H (for Q65), and A, H, L, W and Y (for F67) All mutant proteins were overexpressed mainly as a soluble form in a similar amount to that of the wild-type protein (FIGS. 2, 3, and 4; 126 mg/L culture). The behaviors in the affinity column were observed to be quite similar. The side chains of these amino acids are arranged to point to the solvent and these amino acids belong to the intrinsically flexible region of the Erm protein. The effects of the introduced mutation on the activity of ErmS in cells and in vitro were analyzed.

In vivo activity of ErmS and mutant proteins thereof (antibiotic sensitivity analysis)

8 µl of erythromycin stock solution (25 mg/mL) was dropped onto a Whatmann 3 MM paper punched in a form of a disc until a final amount of 200 µg was reached and dried in the air. E. coli cultures were transferred to fresh LB medium (10%, v/v) and incubated at 37° C. for another 1.5 hours to reach an A₆₀₀ of 0.8 to 1.0, and then spread on a pre- warmed LB agar plate with cotton swabs. To test antibiotic resistance, the dried disk-form Whatmann papers containing erythromycin were placed in the center of the culture of antibiotic susceptible strain (harboring pET23b), the culture of resistant strain (harboring pHJJ105) and the culture of test strain (expressing mutant proteins). The cultures were incubated overnight at 37° C. and then inhibition zones were examined, which are formed due to retarded growth by the inhibitory action of erythromycin.

### In Vitro Methylation Analysis

In vitro methylation of Bacillus subtilis 23S rRNA domain V (BDV, 668 nt) by Erm protein was performed with modification of the previously disclosed procedure (Protein Expr. Purif. 2002, 25, 149-159, J. Bacteriol. 1994, 176, 6992-6998, [symbol] J. Bacteriol. 1995, 177, 4327-4332). The reaction was carried out in a 50 µl reaction volume including 50 mM Tris-HCl (pH 7.5), 4 mM MgCl₂, 40 mM KC1, 10 mM dithiothreitol (DTT), 3.3 pmol S-adenosyl-L-methionine (SAM; specific activity, 80 Ci/mmole; PerkinElmer), 10 pmol rRNA transcript, and 6.76 pmol (250 ng) purified Erm protein. The reaction mixture containing all components except for proteins was pre-warmed by incubation at 37° C. for at least 5 minutes, and purified Erm proteins were added to the warmed tube to minimize delay to initiation of the reaction. After 1 hour of incubation, 0.5 mL of ice-cold 12% trichloroacetic acid (TCA) was added to terminate the reaction. Methylated RNA recovered by centrifugation was washed twice with 1.25 mL of ice-cold 6% TCA. After drying, the precipitate was extracted with 3 mL of scintillation fluid (Ultima Gold; Packard), transferred to a counting vial and counted (Tri-Carb 2900TR; Packard, Shelton, CT, USA). Experiments were repeated at least three times.

### S64 Mutant

When amino acids having a side chain larger than that of serine (threonine, phenylalanine, and tyrosine) were introduced, the activity of inducing resistance to erythromycin in cells was lost. This was also confirmed by the in vitro activity of the purified protein. Tables 1 and 2 below show the results of in vitro methylation activity analysis. However, the enzymatic activity of the mutants with side chains similar to or smaller than that of S64 protected the cells without inhibition zone around the disc containing 200 µg of erythromycin, whereas E. coli expressing inactive mutant Erm protein or E. coli having no Erm showed a measurable inhibition zone (FIG. 2). In vivo activity of each mutant for domain V was measured, in order to characterize the S64 mutants that retained methyltransferase (MTase) activity to confer resistance to erythromycin. Domain V is a complete substrate known to contain all structural elements for an Erm protein (J. Bacteriol. 1994, 176, 6992-6998, J. Bacteriol. 1994, 176, 6999-7004). The structure is shown in FIG. 5. Mutations S64G, S64A and S64C exhibited 71%, 21% and 20% activity compared to the wild-type, respectively.

TABLE 1

| Protein | Methylation activity (cpm) |
|---|---|
| Wild-type | 16,922 ± 197 |
| S64A | 3703 ± 172 |
| S64C | 3523 ± 184 |
| S64G | 12,050 ± 119 |
| S64F | 101±17 |
| S64T | 76 ± 20 |
| S64Y | 86 ± 22 |
| Control group (no protein) | 82 ± 23 |

### Q65 Mutant

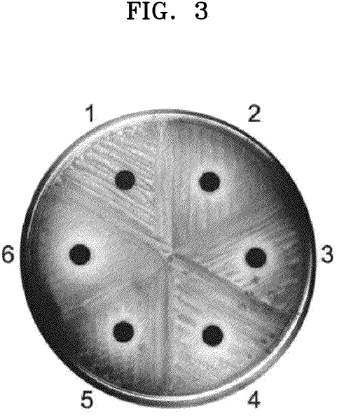

When the Q65N mutation was introduced into ErmS and expressed in E. coli, resistance to erythromycin was lost and a sensitive phenotype was exhibited. In addition, cells expressing mutations such as Q65E, Q65R, and Q65H were also found to form an inhibition zone around the disc containing 200 µg of erythromycin. Even purified mutant proteins were found to lose the activity of transferring a methyl group to domain V in an in vitro methylation analysis. The results are shown in Table 2 and FIG. 3 below.

TABLE 2

| Protein | Methylation activity (cpm) |
|---|---|
| Wild-type | 16,922 ± 197 |
| Q65E | 131 ± 28 |
| Q65N | 108±22 |
| Q65R | 58± 27 |
| Q65H | 70 ± 23 |
| Control group (no protein) | 82 ± 23 |

### F67 Mutant

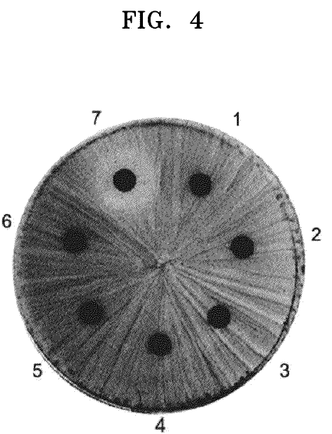

In contrast to Q65 mutants, all mutations for F67, whether conservative or non-conservative, were found to confer resistance to erythromycin. However, in vitro methylation activity varied depending on the nature of the side chain that is present in the place of the phenylmethyl group of phenylalanine. When the size of the side chain is reduced to alanine or when a positive charge is introduced, the imidazole ring in histidine may decrease the methylation activity by about 4%. However, increasing the size of the side chain from methyl (alanine) to isobutyl (leucine) enhanced the activity more than 10 times compared to that of F67A and F67H. The introduction of a larger side chain than phenylalanine (W and Y) may reduce the activity to about one-fourth of the wild-type methylation activity. The results are shown in Table 3 and FIG. 4.

TABLE 3

| Protein | Methylation activity (cpm) |
|---|---|
| Wild-type | 16,922 ± 197 |
| F67A | 700±34 |
| F67H | 735±15 |
| F67L | 7202±103 |
| F67W | 4675±103 |
| F67Y | 4069±135 |
| Control group (no protein) | 82 ± 23 |

Tables 4 to 7 below show the DNA oligonucleotide primers used to clone DNA fragments encoding various ermS mutants.

TABLE 4

| | | |
|---|---|---|
| Name (SEQ ID NO) | Sequence (5′ to 3′) | Description |
| Oligo-1 (SEQ ID NO: 1) | GGAATTCCATATGGCTCGTGCA CCGCGTTCT | 31-mer forward primer for ermS cloning, containing a restriction enzyme recognition site overlapping the initiation Met codon (NdeI, bold) |
| Oligo-2 (SEQ ID NO: 2) | CCCAAGCTTCCGTCCGGCCGGT CGGCT | 27-mer reverse primer containing restriction enzyme recognition site (HindIII, bold) |

TABLE 5

| | | |
|---|---|---|
| Name (SEQ ID NO) | Sequence (5′ to 3′) | Description |
| Oligo-3 (SEQ ID NO: 3) | CGCGAGCTCGCTCAGAAC TTCCTCG CC | 27-mer forward primer for S64A cloning |
| Oligo-4 (SEQ ID NO: 4) | GAAGTTCTGAGCGAGCTC GCGCCGC GC | 27-mer reverse primer for S64A cloning |
| Oligo-5 (SEQ ID NO: 5) | CGCGAGCTCTGCCAGAAC TTCCTCG CC | 27-mer forward primer for S64C cloning |
| Oligo-6 (SEQ ID NO: 6) | GAAGTTCTGGCAGAGCTC GCGCCGCGC | 27-mer reverse primer for S64C cloning |
| Oligo-7 (SEQ ID NO: 7) | CGCGAGCTCGGTCAGAAC TTCCTCG CC | 27-mer forward primer for S64G cloning |
| Oligo-8 (SEQ ID NO: 8) | GAAGTTCTGACCGAGCTC GCGCCGCGC | 27-mer reverse primer for S64G cloning |
| Oligo-9 (SEQ ID NO: 9) | CGCGAGCTCTTCCAGAACT TCCTCG CC | 27-mer forward primer for S64F cloning |
| Oligo-10 (SEQ ID NO: 10) | GAAGTTCTGGAAGAGCTC GCGCCGCGC | 27-mer reverse primer for S64F cloning |
| Oligo-11 (SEQ ID NO: 11) | CGCGAGCTCACCCAGAAC TTCCTCG CC | 27-mer forward primer for S64T cloning |
| Oligo-12 (SEQ ID NO: 12) | GAAGTTCTGGGTGAGCTC GCGCCGC GC | 27-mer reverse primer for S64T cloning |
| Oligo-13 (SEQ ID NO: 13) | CGCGAGCTCTACCAGAACT TCCTCGCC | 27-mer forward primer for S64Y cloning |
| Oligo-14 (SEQ ID NO: 14) | GAAGTTCTGGTAGAGCTC GCGCCGCGC | 27-mer reverse primer for S64Y cloning |

TABLE 6

| | | |
|---|---|---|
| Name (SEQ ID NO) | Sequence (5′ to 3′) | Description |
| Oligo-15 (SEQ ID NO: 15) | GAGCTCTCGGAAAACTTCCTC GCCCGC | 27-mer forward primer for Q65E cloning |
| Oligo-16 (SEQ ID NO: 16) | GAGGAAGTTTTCCGAGAGCTC GCGCCG | 27-mer reverse primer for Q65E cloning |
| Oligo-17 (SEQ ID NO: 17) | GAGCTCTCGAACAACTTCCTCGC CCGC | 27-mer forward primer for Q65N cloning |
| Oligo-18 (SEQ ID NO: 18) | GAGGAAGTTGTTCGAGAGCTCGC GCCG | 27-mer forward primer for Q65N cloning |
| Oligo-19 (SEQ ID NO: 19) | GAGCTCTCGAGAAACITCCTCGC CCGC | 27-mer forward primer for Q65R cloning |
| Oligo-20 (SEQ ID NO: 20) | GAGGAAGTTTCTCGAGAGCTC GCGCCG | 27-mer reverse primer for Q65R cloning |
| Oligo-21 (SEQ ID NO: 21) | GAGCTCTCGCACAACTTCCTCG CCCGC | 27-mer forward primer for Q65H cloning |
| Oligo-22 (SEQ ID NO: 22) | GAGGAAGTTGTGCGAGAGCTC GCGCCG | 27-mer reverse primer for Q65H cloning |

TABLE 7

| | | |
|---|---|---|
| Name (SEQ ID NO) | Sequence (5′ to 3′) | Description |
| Oligo-23 (SEQ ID NO: 23) | TCGCAGAACGCACTCGCCCG CCGGGCC | 27-mer forward primer for F67A cloning |
| Oligo-24 (SEQ ID NO: 24) | GCGGGCGAGTGCGTTCTGCG AGAGCTC | 27-mer reverse primer for F67A cloning |
| Oligo-25 (SEQ ID NO: 25) | TCGCAGAACCACCTCGCCCG CCGGGCC | 27-mer forward primer for F67H cloning |
| Oligo-26 (SEQ ID NO: 26) | GCGGGCGAGGTGGTTCTGCG AGAGCTC | 27-mer reverse primer for F67H cloning |
| Oligo-27 (SEQ ID NO: 27) | TCGCAGAACCTGCTCGCCCG CCGGGCC | 27-mer forward primer for F67L cloning |
| Oligo-28 (SEQ ID NO: 28) | GCGGGCGAGCAGGTTCTGCG AGAGCTC | 27-mer reverse primer for F67L cloning |
| Oligo-29 (SEQ ID NO: 29) | TCGCAGAACTGGCTCGCCCG CCGGGCC | 27-mer forward primer for F67W cloning |
| Oligo-30 (SEQ ID NO: 30) | GCGGGCGAGCCAGTTCTGCG AGAGCTC | I 27-mer reverse primer for F67W cloning |
| Oligo-31 (SEQ ID NO: 31) | TCGCAGAACTACCTCGCCCGC CGGGCC | 27-mer forward primer for F67Y cloning |
| Oligo-32 (SEQ ID NO: 32) | GCGGGCGAGGTAGTTCTGCG AGAGCTC | 27-mer reverse primer for F67Y cloning |

In order to obtain a mutant ermS gene by SOE-PCR (splicing by overlap extension PCR), 4 oligonucleotide primers are required. Two oligonucleotide primers are for the N-terminus and the C-terminus, and the other two are for the intermediate region including the site to be mutated. The oligonucleotide primers for the intermediate region should contain overlapping regions to be extended and amplified by the oligonucleotide primers for the N-terminal and the C-terminal (oligo-1 and oligo-2). The underlined sequences in oligo-3 to oligo-32 indicate overlapping sequences for extension.

1. A method of screening an inhibitor of an erythromycin ribosome methylation (Erm) protein that induces resistance to macrolide-licosamide-streptogramin B (MLS_{B}) antibiotics, comprising: designing a substance that induces a conformational change in a region including an N-terminal end region (NTER) and/or a shortest motif X adjacent thereto, S—Q—N—F, of an Erm protein; contacting the substance with an Erm protein or a complex of an Erm protein and an RNA substrate and/or a ligand to select a candidate substance that induces a conformational change in a region containing the NTER and/or the shortest motif X adjacent thereto and/or a site that binds to the substrate or the ligand in the Erm protein or the complex together with the motif X, NTER, or a complex thereof, and identifying whether the conformational change leads to inhibition or elimination of the methylation activity of the Erm protein.

2. The method of claim 1, wherein the selecting a candidate substance is performed in silico.

3. The method of claim 1, wherein the selecting a candidate substance is performed by using one or more of X-ray crystallography, cryogenic electron microscopy (cryo-EM), and nuclear magnetic resonance (NMR) spectroscopy.

4. The method of claim 1, wherein the inhibition or elimination of the methylation activity is determined by an in vitro methylation activity assay, an in vivo antibiotic resistance assay, or a minimal inhibitory concentration (MIC) measurement.

5. The method of claim 1, wherein the candidate substance is a compound, a nucleic acid, a peptide, or a protein.

6. The method of claim 1, wherein the conformational change is change in a length of the NTER of the Erm protein or a distance between the Erm protein and/or the RNA substrate or ligand.

7. The method of claim 6, wherein the candidate substance causes a conformational change in the shortest motif X, S—O—N—F by changing the distance between the nitrogen of the carbamoyl group in Q and oxygen of a hydroxyl group in S the distance between the nitrogen of the carbamoyl group in Q and C4 carbon of the phenyl group in F. the distance between the oxygen of the side chain hydroxyl group in S and the 6th nitrogen in the adenine, a methylation target.

8. The method of claim 1, wherein the Erm protein is ErmC’ or ErmS, and the RNA substrate is 23S rRNA, domain V, or a fragment thereof.

9. The method of claim 1, further comprising applying the candidate substance identified to lead to inhibition or elimination of the methylation activity to an in vivo antibiotic resistance assay or to a minimal inhibitory concentration (MIC) measurement.

10. An inhibitor of an Erm protein, which reduces or eliminates methylation activity of an Erm protein by binding to the Erm protein to lead to conformational or functional change in an NTER and/or a shortest motif X adjacent thereto, S—Q—N—F.

11. The inhibitor of claim 10, wherein the inhibitor introduces an amino acid substitution into the shortest motif X.

12. The inhibitor of claim 11, wherein the substitution is substitution of S with G, A, C, T, F, or Y, substitution of Q with N, E, R, or H, or substitution of F with A, H, L, Y, or W in the shortest motif X.

13. The inhibitor of claim 11, wherein the substitution is a substitution of at least 2 amino acids in the SQNF motif.

14. An antibacterial composition comprising the inhibitor of claim 10 and an MLS_{B}.
